(19) United States
(12) Patent Application Publication
(10) Pub. No.: US 2026/0260714 A1
(43) Pub. Date: Sep. 3, 2026

(54) IDENTIFYING A SCALE INHIBITOR USING A PHYSICS-BASED MODEL AND A MACHINE LEARING MODEL

(71) Applicant: Schlumberger Technology Corporation, Sugar Land, TX (US)

(72) Inventors: Clare Schoene, Houston, TX (US); Kang Wang, Houston, TX (US); Erin Temple, Aberdeen (GB); Chunfang Fan, Houston, TX (US); Alexander Colin Nelson, Stavanger (NO); Tiffany Nguyen, Dubai (AE)

(21) Appl. No.: 19/068,171

(22) Filed: Mar. 3, 2025

(51) Int. Cl. G16C 20/30; G16C 20/20; G16C 20/70

(52) U.S. Cl. CPC G16C 20/30; G16C 20/20; G16C 20/70

(57) ABSTRACT

A model may identify a scale type for the target water-bearing fluid using a physics-based model. A model may input one or more known properties of the target water-bearing fluid and the scale type into a machine learning model. A model may extrapolate unknown properties of a historical data set using the machine learning model. A model may predict a scale inhibitor applicability index based on the historical data set and extrapolated unknown properties for the plurality of scale inhibitors. A model may output one or more of a list of scale inhibitors for use with the target water-bearing fluid or a list of scale inhibitor properties.

### BACKGROUND OF THE DISCLOSURE

Produced water, a byproduct of oil and gas production, may be rich in minerals from the formation. The water may have a high tendency to form inorganic deposits, known as scale, as the pressure and temperature of the water change during extraction and transportation. Scale can deposit in the wellbore on screens, pumps, valves, separators, piping, subsurface and surface equipment, and pipelines. Scale deposits can reduce production rates and cause equipment failure ultimately leading to production losses.

Typically, a scale inhibitor may be injected into fluid processing and transportation systems at various locations. The water chemistry of each well is unique and selection of the correct scale inhibitor for a specific water chemistry is an important step in designing a scale management program. Conventional scale inhibitor selection is time-intensive and requires time consuming laboratory tests on multiple inhibitors chosen by a subject matter expert, to be well suited for the water chemistry among other operational requirements such as operating conditions and compatibility.

### SUMMARY

In some aspects, the techniques described herein relate to a method for selecting scale inhibitors for a target water-bearing fluid. A scale inhibitor selection system identifies a scale type for the target water-bearing fluid using a physics-based model. One or more known properties of the target water-bearing fluid and the scale type are input into a machine learning model. The machine learning model extrapolates unknown properties of a historical data set using the machine learning model. The machine learning model is trained on the historical data set including one or more properties of a plurality of water-bearing fluids, one or more properties of a plurality of scale inhibitors to inhibit the scale type identified by the physics-based model, and one or more scale inhibitor applicability indexes of the scale inhibitors with the plurality of water-bearing fluids. The machine learning model predicts a scale inhibitor applicability index based on the historical data set and extrapolated unknown properties for the plurality of scale inhibitors. The machine learning model outputs one or more of a list of scale inhibitors for use with the target water-bearing fluid or a list of scale inhibitor properties.

In some aspects, the techniques described herein relate to a method including. A scale inhibitor selection system receives water properties of a fluid sample of a water-bearing fluid. The scale inhibitor selection system applies a physics-based model to the water properties. The physics-based model identifies a scale type and a saturation index of the scale type for the fluid sample under a specific temperature and pressure condition or a range of temperatures and pressures. A machine learning model is applied to the water properties, the scale type, the saturation index, and system conditions. The machine learning model is trained to generate an output of at least one scale inhibitor based on an input of water properties and selection conditions including but not limited to system temperature & pressure, application types, material compatibility, brine compatibility, chemical compatibility, global/regional regulatory requirements, and HSE requirements. The machine learning model outputs a list of scale inhibitors for the fluid sample based on the water properties, the scale type and the saturation index, and other selection conditions listed before.

This summary is provided to introduce a selection of concepts that are further described in the detailed description. This summary is not intended to identify key or essential features of the claimed subject matter, nor is it intended to be used as an aid in limiting the scope of the claimed subject matter. Additional features and aspects of embodiments of the disclosure will be set forth herein, and in part will be obvious from the description, or may be learned by the practice of such embodiments.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a schematic representation of a scale inhibitor selection system, according to at least one embodiment of the present disclosure.

FIG. 2 is a flow diagram of a scale inhibitor selection system, according to at least one embodiment of the present disclosure.

FIG. 3 is a flow diagram of a scale inhibitor selection system, according to at least one embodiment of the present disclosure.

FIG. 4 shows a schematic view of a system to facilitate the selection of a suitable scale inhibitor for use with a specific water-bearing fluid, according to at least one embodiment of the present disclosure.

FIG. 5 is a flowchart of a method for selecting scale inhibitors, according to at least one embodiment of the present disclosure.

FIG. 6 is a flowchart of a method for selecting scale inhibitors, according to at least one embodiment of the present disclosure.

FIG. 7 is a representation of a computing system, according to at least one embodiment of the present disclosure.

### DETAILED DESCRIPTION

This disclosure generally relates to devices, systems, and methods for identifying and recommending scale inhibitors that may be compatible with a target water-bearing fluid. Scale includes the deposition of one or more compounds from water. Scale deposition may occur when the concentration of dissolved ions exceeds a saturation level, thereby causing the formation of a solid compound. For example, scale deposition may occur when the thermodynamic conditions of the water-bearing fluid change, thereby changing the saturation level of the water for a particular compound. Scale deposition may clog pipelines and/or pipeline equipment, such as valves, pumps, and the like. Scale deposition may be prevented using a scale inhibitor. The scale inhibitor may include a chemical added to the water-bearing fluid that reduces or prevent the precipitation of the compounds that form the scale.

Scale inhibitors are typically identified by testing multiple products, and then selecting the scale inhibitor with the best performance. However, such testing is costly and time-consuming. The scale inhibitors tested may be selected based on information in a product catalog, combined with a technician's knowledge and expertise. This may result in the operator testing an excessive number of products. The results of various scale inhibitor tests may be stored in a test database, resulting in a large amount of historical data. But due to the number of products to be tested, it may be difficult to identify the most effective scale inhibitor.

In accordance with at least one embodiment of the present disclosure, a scale inhibitor selection system may extrapolate the historical data into a form that allows reliable predictions to be made about the interactions between water-bearing fluids and different scale inhibitors. The scale inhibitor selection system may identify correlations between the properties of water in the water-bearing fluid and the properties of scale inhibitors.

In some embodiments, a physics-based model may identify a scale type that is likely to form based on the properties of the water in the water-bearing fluid. For example, the physics-based model may predict and identify mineral scale types and thermodynamic saturation levels based on cationic and anionic composition and system conditions. The physics-based model may generate a saturation index for a particular scale type. The saturation index may be a representation of the saturation of a particular compound with respect to a type of scale.

A method for selecting scale inhibitors that have a high scale inhibitor applicability index for a target water-bearing fluid may include a user inputting one of more known properties of a target water-bearing fluid into a machine learning model that has been trained by a historical data set that includes one or more properties of a plurality of water-bearing fluids, one or more properties of a plurality of scale inhibitors, the scale type, the saturation index for the scale type, and the compatibility of the scale inhibitors with the associated water-bearing fluids. Historical data also may include testing conditions of scale inhibitors with water-bearing fluids thermodynamic properties (e.g., pressure and temperature), the testing apparatus used, and set-up and take down procedures of each test. The historical data set includes test results of a combination of a water-bearing fluid and a scale inhibitor. The machine learning model extrapolates unknown properties (e.g., unknown correlations between parameters) of the target water-bearing fluid and data sets within the historical data set, such as connections or relationships between the water-bearing fluid and properties of the scale inhibitor. The machine learning model predicts an applicability index based on the historical data set and extrapolated unknown properties for the one or more scale inhibitors that may be used with the target water-bearing fluid. The machine learning model outputs one or more of a list of water-bearing fluids having one or more properties within a numerical tolerance of the properties of the target water-bearing fluid, a list of scale inhibitors for use with the target water-bearing fluid; a list of scale inhibitor properties; and a compatibility of the proposed scale inhibitors with the target water-bearing fluid.

A user or the machine learning model may select or design a chosen scale inhibitor, based on the output, for use with the target water-bearing fluid. In one embodiment, the user may test a plurality of the output scale inhibitors with the target water-bearing fluid. Based on the actual test data, the user selects the scale inhibitor to be used with the target water-bearing fluid. Once selected, a user may validate the predicted scale inhibitor applicability index with testing on the scale inhibitor and the target water-bearing fluid, and add the results of the actual testing to the historical data set.

The one or more properties of a plurality of water-bearing fluids may include a location of a source of one of the plurality of water-bearing fluids. The location of a source of a water-bearing fluid may allow the machine learning model to identify additional data sets within the historical data that may be closely related to the water-bearing fluid and facilitate the identification of effective scale inhibitors. The one or more properties of a plurality of water-bearing fluids may also include dissolved ions in the water-bearing fluids, such as sodium (Na), potassium (K), magnesium (Mg), calcium (Ca), Strontium (Sr), barium (Ba), iron (Fe), zinc (Zn), lead (Pb), chloride (Cl⁻), sulphate (SO₄²⁻), fluoride (F⁻), bromide (Br⁻), silica (SiO₂), bicarbonate (HCO₃⁻). In some embodiments, the properties may include total dissolved solids (TDS), carbon dioxide (CO₂) gas concentration, hydrogen sulfide (H₂S) gas concentration, pH, and so forth.

The one or more properties of a plurality of scale inhibitors may include chemistry type, composition, raw material code, type of scale that it treats, regional availability, environmental data, viscosity, thermal stability, calcium tolerance, freeze point, flash point, and so forth. Similarly, the molecular properties of scale inhibitors may facilitate the machine learning model in identifying and building correlation models to improve its ability to predict the structure and composition of an effective scale inhibitor with a specific water-bearing fluid.

As part of the method, the machine learning model may calculate a scale inhibitor applicability index for each combination of a scale inhibitor and the target water-bearing fluid using the historical data set. Prior to the outputting step, the machine learning model may order the scale inhibitors for use with the target water-bearing fluid based on the predicted scale inhibitor applicability index. As used herein, the term “ordering” means to arrange in a methodical way and should be interpreted to include the ranking of scale inhibitors by scale inhibitor applicability. For example, ordering the scale inhibitors may mean placing the most applicable scale inhibitor at the top of an outputted list. Alternatively, ordering the scale inhibitors may mean merely indicating a set of scale inhibitors have good applicability index compared to other scale inhibitors that have poor applicability index with a target water-bearing fluid.

In some embodiments, a user or a machine learning model may add, to a historical database, records of water-bearing fluids and scale inhibitors. Each record includes one or more of properties of the water-bearing fluid including identification of a source of a water-bearing fluid, scale content, scale inhibitor identification, scale type, scale saturation index, field operating conditions, and so forth. The machine learning model generates first model parameters to compare a target water-bearing fluid to the water-bearing fluids in the database. The machine learning model generates second model parameters to compute a scale inhibitor efficiency of the scale inhibitors in the database with the target water-bearing fluid and generates third model parameters to compute an applicability index of the scale inhibitor in the database with the target water-bearing fluid. The machine learning model inputs properties of the target water-bearing fluid into a model containing the first model parameters, the second model parameters, and the third model parameters. The machine learning model then outputs one or more of a list of water-bearing fluids having one or more properties within a numerical tolerance of the properties of the target water-bearing fluid, a list of scale inhibitors for the target water-bearing fluid, each including a scale inhibitor applicability index, and a list of inhibitor properties.

The machine learning model or a user may select or design a chosen scale inhibitor, based on the output, for use with the target water-bearing fluid. In one embodiment, the user may test a plurality of the output scale inhibitors with the target water-bearing fluid. Based on the actual test data, the user selects the scale inhibitor to be used with the target water-bearing fluid. Once selected, a user may validate the confidence level with testing on the scale inhibitor and the target water-bearing fluid and add the results of the actual testing to the historical data set. The historical data set may include test results of a combination of a water-bearing fluid and a scale inhibitor.

As illustrated by the foregoing discussion, the present disclosure utilizes a variety of terms to describe features and advantages of the scale inhibitor selection system. Additional detail is now provided regarding the meaning of such terms. For example, as used herein, a “water-bearing fluid” may include a substance that is collected from an oil and gas system. For instance, a “water-bearing fluid” may include a single phase of matter, such as a gas or a liquid. In some embodiments, a water-bearing fluid may include a combination of multiple materials and/or multiple phases of matter, such as a liquid having suspended solids, a liquid having entrained gas or solids, a liquid having gas bubbles, a gas suspended liquid droplets or solid particles. The water-bearing fluid may include a combination two or three of water (e.g., a brine including dissolved compounds), oil (e.g., crude oil), gas (e.g., natural gas), including a mixture of water and oil, a mixture of water and gas, or a mixture of water, oil, and gas. The water-bearing fluid may include one or more compounds, including compounds dissolved in water, a mixture of different hydrocarbons, compounds dissolved in crude oil, or a mixture of two or more gasses.

As used herein, the term “scale” may represent solid material that has been deposited on the surface of a container in which a water-bearing fluid has been stored and/or transported. Scale may form through precipitation of dissolved ions or compounds in a water-bearing fluid. Precipitation of scale may occur based on changes in thermodynamic conditions and/or changes in the chemistry of the water-bearing fluid. Compounds that form scale may include calcium carbonate, calcium sulfate (gypsum and anhydrites), barium sulfate, strontium sulfate, iron sulfide, iron carbonate, zinc carbonate, zinc sulfide, lead sulfide, sodium chlorides, and silicate based on the cationic and anionic composition of water.

As used herein, the term “scale inhibitor” may refer to a chemical, compound, or additive that may be added to a water-bearing fluid to reduce the likelihood of scale formation, or prevent altogether scale formation. A scale inhibitor may reduce the likelihood of scale formation by altering the pH of the water-bearing fluid, adjusting the chemical equilibrium of the water-bearing fluid, or otherwise altering the water-based fluid.

As used herein, the term “machine learning” refers to algorithms that generate data-driven predictions or decisions from known input data by modeling high-level abstractions. Examples of machine-learning models include computer representations that are tunable (e.g., trainable) based on inputs to approximate unknown functions. For instance, a machine-learning model includes a model that utilizes algorithms to learn from, and make predictions on, known data by analyzing the known data to learn to generate outputs that reflect patterns and attributes of the known data. For example, machine-learning models include latent Dirichlet allocation (LDA), multi-arm bandit models, linear regression models, classification models, logistical regression models, random forest models, support vector machines (SVMs) models, neural networks (convolutional neural networks, recurrent neural networks such as LSTMs, graph neural networks, etc.), or decision tree models.

A machine learning model may be tuned (e.g., trained) based on training input to approximate unknown functions. For example, a machine learning model may refer to a neural network or other machine learning algorithm or architecture that learns and approximates complex functions and generate outputs based on a plurality of inputs provided to the machine learning model. In some embodiments, a machine learning model may include one or multiple machine learning models that cooperatively generate one or more outputs based on corresponding inputs.

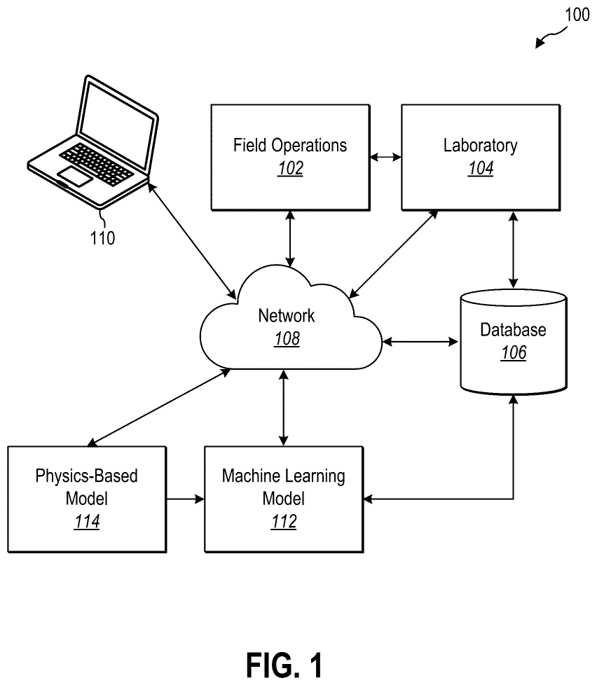

FIG. 1 is a schematic representation of a scale inhibitor selection system 100, according to at least one embodiment of the present disclosure. The scale inhibitor selection system 100 may facilitate the identification and selection of a scale inhibitor. Fluid samples may be collected from one or more field operations 102. One or more of the fluid samples may include a water-bearing fluid. The field operations 102 may include any field operation in an oil and gas exploration and production system. For example, the field operations 102 may include wellbore drilling, wellbore completion, wellbore production, wellbore pipelines and associated equipment, any other field operations, and combinations thereof. An operator may collect fluid samples from the field operations 102. For example, the operator may collect a fluid sample of fluids pumped into and subsequently retrieved from a wellbore (e.g., drilling fluid or drilling mud, hydraulic fracturing fluid, artificial lift fluid). In some examples, the operator may collect a fluid sample of fluids produced by a wellbore, such as water and/or oil.

A laboratory 104 may receive the fluid sample from the field operations 102. The laboratory 104 may perform one or more tests on the fluid. For example, the laboratory 104 may perform tests to determine the composition of the fluid, the physical properties of the fluid, the chemical properties of the fluid, and so forth. In some embodiments, the laboratory 104 may determine the likelihood that scale will form or deposit based on the water-bearing fluid. In some examples, the laboratory 104 may analyze the impact of one or more additives on the fluid. For example, the laboratory 104 may analyze the impact of adding a scale inhibitor

In some embodiments, a single laboratory 104 may analyze the fluid sample collected from the field operations 102. In some embodiments, the field operations 102 may send the fluid sample to multiple laboratories 104, with different laboratories 104 performing different tests on the fluid sample.

The laboratory 104 may generate one or more lab reports associated with the tests performed on the fluid sample. The lab reports may include data stored in one or more data fields. For example, the lab reports may include the testing conditions, the properties of the fluid sample, the scale inhibitor tested, the testing results, and information associated with the source of the sample. In some embodiments, the field operations 102 may include field operating conditions, or operational information associated with collecting the fluid sample, such as temperatures, pressures, wellbore identification, wellbore location, relevant geology, collection equipment, collection crew, wellbore owner information, and so forth.

As discussed herein, the lab reports may be generated to information operational decisions. For example, the lab reports may be generated to inform the selection of an additive to a fluid stream. Typically, when an operator desires to make an operational decision, the operator may request the collection and analysis of the fluid samples. However, such analysis may be expensive and time consuming. The operator may desire to make the operational decision based tests previously performed on other fluid samples, including fluid samples from similar wellbores and/or dissimilar wellbores. Such historical data may be stored in a database 106. The database 106 may include lab reports from various laboratories 104.

The field operations 102, the laboratory 104, and the database 106 may be connected over a network 108, such as the internet. For example, the field operations 102 may collect the sample, record information associated with the collection of the sample in a field report, and upload the field report to the database 106 over the network 108. The field operations 102 may physically deliver the fluid sample to the laboratory 104, and the laboratory 104 may generate the lab report and upload the lab report to the database 106 over the network 108. In some embodiments, the database 106 may be located on a remote server, such as a cloud server, and the field operations 102 and/or the laboratory 104 may upload the lab reports in real time to the database 106. In some embodiments, the field operations 102 and/or the laboratory 104 may upload the lab reports to the database 106 after completion of multiple lab reports, or when internet access is available to the operator. A user may access the lab reports from the database 106 using a user device 110 via the network 108. In some embodiments, the user may further be in communication with the field operations 102 and/or the laboratory 104 over the network 108.

In accordance with at least one embodiment of the present disclosure, a machine learning model 112 may prepare an output of a recommended scale inhibitor based on an inputted target water-bearing fluid. For example, the machine learning model 112 may be trained based on historical data from the database 106. The machine learning model 112 may identify or extrapolate unknown properties of the historical data in the database 106, including correlations between scale inhibitors, water properties, and field operating conditions.

In accordance with at least one embodiment of the present disclosure, the scale inhibitor selection system 100 may further include a physics-based model 114. The physics-based model 114 may identify scale types and prepare a saturation index for the water-bearing fluid based on the water properties from the fluid sample. For example, the physics-based model 114 may predict and identify mineral scale types and thermodynamic saturation levels based on cationic and anionic composition and system conditions (e.g., pressure, temperature, and pH). The resulting scale type and saturation index may be provided to the machine learning model 112 as additional input. For example, the scale type and saturation index may be derived or engineered features used during the training of the machine learning model 112 and/or when applying the machine learning model 112 to the water-bearing fluid sample. This may improve the accuracy and/or relevance of the output of the machine learning model 112.

In some embodiments, the database 106 may be updated with the derived features of from the physics-based model 114. For example, the physics-based model 114 may be applied to the historical data in the database 106. The database 106 may be updated with the resulting scale type and associated saturation index. In this manner, the machine learning model 112 may be trained based on the derived features stored in the updated database 106.

The physics-based model 114 may predict and identify the types of mineral scales that could form in the saturated water based on chemical ion compositions and field operating conditions (such as temperature and pressure). The physics-based model 114 may calculate the thermodynamic saturation levels of the occurring mineral scales based on the cationic and anionic composition of water. The predictions provide an indication of scale type and risk of precipitation with changes in production conditions. The results of the scale prediction amounts may be presented in the form of saturation index and mass of precipitation based on the complete water analysis at the pH, temperature, and pressure conditions at the time or point of interest.

The saturation index is a measure of the degree of supersaturation of the scaling ions to form. An increase in saturation index increases the potential for precipitation of the scaling ion of interest. The equilibrium precipitation is defined as the mass of the scale of interest that could potentially precipitate out of the already supersaturated solution. A saturation index greater than zero for each predicted scale indicates supersaturation of the water with respect to the scale of interest. This suggests that scale precipitation for the identified mineral scale is thermodynamically favorable. The saturation index for a specific scale type may be defined by:

$$S I = {log}_{10} ( \frac{I A P}{{K}_{sp}} )$$

Where SI is the saturation index, IAP is ion activity product (e.g., the product of the activities of ions in solution that form the mineral), and Kₛₚ is solubility product (e.g., the equilibrium constant for the mineral's dissolution reaction). The saturation index may be further processed categorically. For example, a saturation index of less than zero indicates that water is undersaturated for the predicted scale type, and therefore unlikely to produce scale. A saturation index of between 0.1 and 1.0 may be representative of scale that is likely to occur, but is not severe. A saturation index of greater than 1.0 may be representative of a solution that is super saturated, which may result in scale precipitation that is almost certain to occur, resulting in high or severe scaling.

While embodiments of the present disclosure may utilize the saturation index to identify the likelihood of scale formation, it should be understood that the saturation ratio may be applied to the techniques of the present disclosure to identify a likelihood of scale formation. The saturation ratio may be a representation of the additional amount of ions in a solution above the solubility product. Put another way, in the context of the SI explained above, the saturation ratio may be IAP divided by K_{SP}.

As discussed herein, the scale type and associated saturation index may be used as derived features for training and/or application of the machine learning model 112. However, it should be understood that the IAP, the Kₛₚ, or other intermediate calculations may be stored or used as derived features for training and/or application of the machine learning model 112. Such derived features may further be stored with the historical data in the database 106.

Other inputs to the machine learning model 112 may include a scale catalog. The scale product catalog may be a representation of the available scale inhibitors. For example, the scale product catalog may include a listing of scale inhibitors and properties thereof. The properties of the scale inhibitor may include chemistry type, composition, raw material code, type of treated scale, regional availability, environmental data, viscosity, thermal stability, calcium tolerance, freeze point, flash point, and so forth. In some embodiments, the properties of the scale inhibitor may be provided by the manufacturer. In some embodiments, the properties of the scale inhibitor may be altered, amended, or otherwise added to by the laboratory 104. In some embodiments, the scale inhibitor selection system 100 may develop one or more derived features based on the interaction between the scale inhibitor properties and the properties of the water samples stored in the database 106. In some embodiments, the historical data in the database 106 may include test results from a particular scale inhibitor, the properties of the scale inhibitor, and the impact of the scale inhibitor on the water sample.

As discussed herein, the machine learning model 112 may be trained based on historical data in the database 106. The machine learning model 112 may be trained to output a list of one or more scale inhibitors. The outputted scale inhibitors may then be applied at the field operations 102 to reduce scale formation and deposition in the oil and gas systems.

In some embodiments, the machine learning model 112 may output multiple recommended scale inhibitors. For example, the machine learning model 112 may generate an applicability index. The applicability index may include an indication of how applicable the associated scale inhibitor is, or be a representation in the level of confidence in the recommendation by the machine learning model 112. In some embodiments, the machine learning model 112 may output all of the scale inhibitors that have an applicability index above a threshold. In some embodiments, the machine learning model 112 may output the scale inhibitors having the highest applicability index. For example, the machine learning model 112 may output the top one, two, three, four, five, six, seven, eight, nine, ten, or more scale inhibitors having the highest applicability index. In some embodiments, the machine learning model 112 may output the single scale inhibitor having the highest applicability index. In this manner, the machine learning model 112 may provide an indication of how applicable or relevant the recommended scale inhibitor are to reducing scale of the water-bearing fluid.

In some embodiments, the machine learning model 112 may further identify a compatibility of the particular scale inhibitor on the water-bearing fluid. For example, the machine learning model 112 may identify, based on the historical data in the database 106, whether a particular scale inhibitor is compatible with the water-bearing fluid. Certain scale inhibitors may have adverse reactions with compounds in the water-bearing fluid. An incompatible scale inhibitor may be a scale inhibitor that adversely impacts the composition or functionality of the water-bearing fluid, such as through undesirable precipitation of compounds or chemical reactions between the scale inhibitor and compounds in the water-bearing fluid.

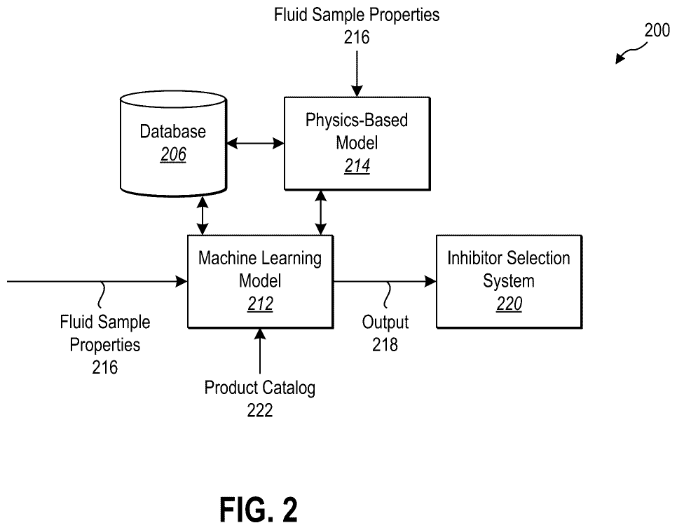

FIG. 2 is a flow diagram of a scale inhibitor selection system 200, according to at least one embodiment of the present disclosure. In the scale inhibitor selection system 200, a machine learning model 212 may receive fluid sample properties 216. The machine learning model 212 may be trained on historical data from a database 206. As discussed herein, the database 206 may include historical lab testing results, including fluid sample properties, test type, tested scale inhibitor, scale inhibitor properties, test results, and so forth. During training, the machine learning model 212 may extrapolate unknown properties of the historical data, which may include connections, relationships, ratios, and so forth between the historical data.

The scale inhibitor selection system 200 may further include a physics-based model 214. As discussed herein, the physics-based model 214 may analyze the fluid sample properties 216, including fluid sample properties in the database 206, to identify or determine the types of scale and the likelihood of the scale forming. For example, the physics-based model 214 may identify the fluid sample properties 216, which may include field operating conditions such as pressure and temperature, and identify, for one or more scale types, the likelihood that scale will form. In some embodiments, the physics-based model 214 may quantify the likelihood that scale will form in a saturation index. In some embodiments, the physics-based model 214 may generate the saturation index for various types of scale for each record of the historical data in the database 206. The physics-based model 214 may further cause the database 206 to be updated with the scale type and the saturation index. The machine learning model 212 may be trained on the updated database 206 having the scale types and associated saturation index. In this manner, the machine learning model 212 may be trained to identify associations between particular scale inhibitors, scale type, and saturation index.

The trained machine learning model 212 may generate an output 218 based on inputted fluid sample properties 216. For example, when the fluid sample properties 216 (e.g., fluid sample properties 216 that are not part of the historical data in the database 206) are input to the machine learning model 212, the machine learning model 212 may generate the output 218 that associates one of or more scale inhibitors with the fluid sample properties 216. The output 218 may be provided to an inhibitor selection system 220. The inhibitor selection system 220 may receive the output 218 and arrange or order the scale inhibitors for selection by the user. For example, the output 218 may include an applicability index and/or a compatibility of the scale inhibitor with respect to the water-bearing fluid that was the source of the fluid sample properties 216. The inhibitor selection system 220 may organize, sort, or otherwise arrange the outputted scale inhibitors to present to a user. In some embodiments, the inhibitor selection system 220 may select the best scale inhibitor to provide to the user.

In some embodiments, the machine learning model 212 may receive, as part of the input, a product catalog 222 of various scale inhibitors. The product catalog 222 may include the identification of scale inhibitors, as well as known properties of the scale inhibitors. In some embodiments, the product catalog 222 may include scale inhibitors that have been used in the recorded historical data of the database 206. In some embodiments, the product catalog 222 may include scale inhibitors that not been used in the recorded historical data of the database 206.

In some examples, the product catalog 222 may be an indication or instruction to the machine learning model 212 of the scale inhibitors that are available. For example, the product catalog 222 may provide an indication of the scale inhibitors that are available within a certain geographical area, available based on supplier relations, available based on stock constraints, or otherwise available.

The output 218 generated by the machine learning model 212 may include any type of scale inhibitor output. For example, the output 218 may include a recommendation of a scale inhibitor that has previously been used in the historical data stored in the database 206. In some embodiments, the output 218 may include a recommendation of a scale inhibitor that has been used in the database 206 and is also provided to the machine learning model 212 as available based on the product catalog 222.

In some embodiments, the machine learning model 212 may provide the output 218 as a set of recommended scale inhibitor parameters. The inhibitor selection system 220 may review the recommended scale inhibitor parameters and select a scale inhibitor from the product catalog 222 based on the recommended scale inhibitor properties. In some embodiments, none of the scale inhibitors from the product catalog 222 are within a particular applicability index range or are compatible with the water-bearing fluid. For unidentified scale inhibitors, the machine learning model 212 may output the list of scale inhibitor properties. A user may request that a manufacturer design or recommend a scale inhibitor that is compliant with the identified properties.

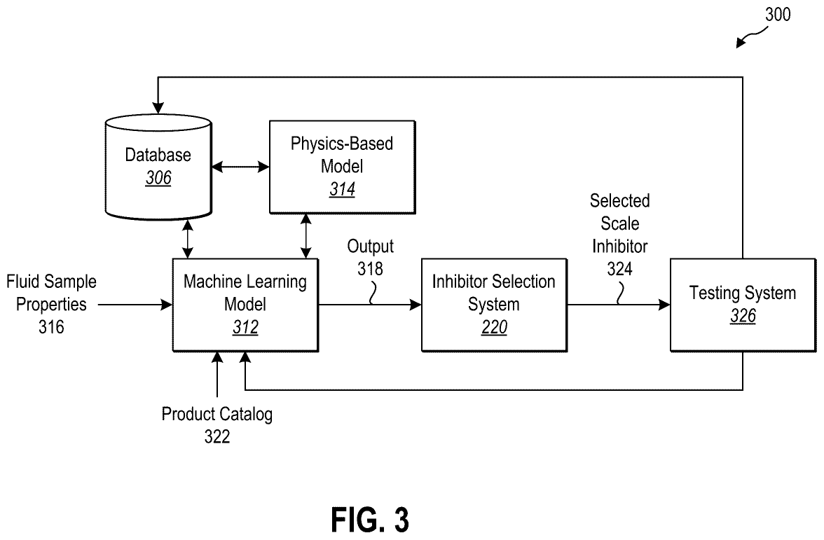

FIG. 3 is a flow diagram of a scale inhibitor selection system 300, according to at least one embodiment of the present disclosure. In the scale inhibitor selection system 300, a machine learning model 312 may receive fluid sample properties 316. The machine learning model 312 may be trained on historical data from a database 306, which may include historical lab testing results, including fluid sample properties, test type, tested scale inhibitor, scale inhibitor properties, test results, and so forth. The scale inhibitor selection system 300 may further include a physics-based model 314. As discussed herein, the physics-based model 314 may analyze the fluid sample properties 316, including fluid sample properties in the database 306, to identify or determine the types of scale and an associated saturation index. The database 306 may include the scale type and the saturation index for various test results.

The trained machine learning model 312 may generate an output 318 based on inputted fluid sample properties 316. For example, when the fluid sample properties 316 (e.g., fluid sample properties 316 that are not part of the historical data in the database 306) are input to the machine learning model 312, the machine learning model 312 may generate the output 318 that associates one of or more scale inhibitors with the fluid sample properties 316. The output 318 may be provided to an inhibitor selection system 320. The inhibitor selection system 320 may receive the output 318 and arrange or order the scale inhibitors for selection by the user.

In some embodiments, one or more selected scale inhibitors 324 may be tested at a testing system 326. For example, the testing system 326 may test the recommended or selected scale inhibitors 324 and determine their applicability to the water-bearing fluid that originated the fluid sample properties 316. As discussed herein, the machine learning model 312 may pre-select certain scale inhibitors. This may reduce the total amount of testing the testing system 326 may need to perform, thereby reducing the time and/or money expended to identify and select a scale inhibitor.

In accordance with at least one embodiment of the present disclosure, the test results from the testing system 326 may be provided to the machine learning model 312 and/or the database 306. The machine learning model 312 may be re-trained and/or fine-tuned based on the test results from the selected scale inhibitors 324 and the fluid sample properties 316. This may help to further improve the accuracy and/or relevance of the results of the machine learning model 312.

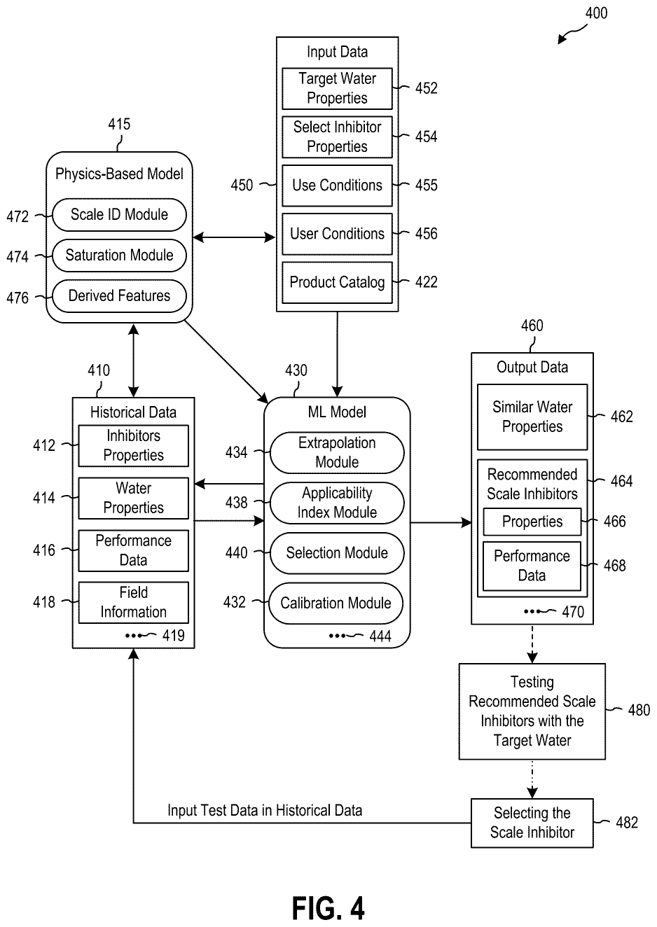

FIG. 4 shows a schematic view of a system 400 to facilitate the selection of a suitable scale inhibitor for use with a specific water-bearing fluid, according to at least one embodiment of the present disclosure. As shown, scale inhibitors properties 412 and the water-bearing fluids properties 414 may be part of a historical database or more generally form part of historical data 410. Additionally, the historical data 410 may include performance data 416, which includes the parameters, procedures, and results of scale inhibitor tests and other water content tests. Performance data 416 may also include the mechanical or crystalline properties of scale formations, including scale formations of water-bearing fluids having scale inhibitors mixed in at different dosages.

Performance data 416 may include a wide variety of experiment procedures and the resulting test data. Test data indicating changes in a deposited mass of scale over time may be included. Unsuccessful test data may also be included in the historical data 410 to provide diverse performance profiles and may allow the model 430 to identify potential areas for tests and data to be gathered.

The historical data 410 may also include field information 418, such as in field reports of scale build up in equipment, journal articles, published information, and the current and historical pricing of scale inhibitors, precursor materials, and manufacturing costs. Anticipated field conditions for use of a scale inhibitor with a target water-bearing fluid may be included to inform selection of test conditions for inclusion in the model 430. Field information 418 may include environmental and field conditions such as water-bearing fluid bulk temperature, pipeline outer wall temperature, weather conditions around a pipeline or production location, flow rates, and pipeline and equipment materials and associated surface friction. Other information 419 not related to the use of scale inhibitors may also be included in the historical data, such as weather reports for specific geographic locations, maps, and geologic formation data.

The historical data 410 may be used to train a model 430. The model 430 uses the historical data 410 to identify potential correlations and similarities in the historical data to make recommendations of potential scale inhibitors that may be used with a target water-bearing fluid. The model 430 may be constructed as a model to which machine learning techniques can be applied. In one embodiment, if the model 430 is to be used to select a list of inhibitors that may be used with a target water-bearing fluid, the model 430 may be a classification type model. If the model is to be used to predict an applicability index for a pairing of a target water-bearing fluid with a selected scale inhibitor, the model 430 may be a regression type model. The model 430 may include modules that perform specific types of evaluations. For example, one module may perform a classification type evaluation while another module performs a regression type evaluation. For example, one module may select a list of inhibitors likely to be successful for a target water-bearing fluid, and another module may predict inhibition efficiency for each of the selected inhibitors with the target water-bearing fluid. For example, the model can contain any or all of a KNN algorithm, a random forest algorithm, a Bayesian network algorithm, a response surface algorithm, a fractional factorial algorithm, or other similar algorithms.

As shown, the model 430 may include multiple modules, including an extrapolation module 434, an applicability index module 438, a selection module 440, a calibration module 442 and other modules 444. The model 430 may be a machine learning model, a large language model, or use artificial intelligence to train itself on the historical data 410 to identify potential correlations between the scale inhibitors properties 412 and the water-bearing fluids properties 414 using the performance data 416, and in some embodiments, the field information 418.

The extrapolation module 434 of the model 430 may extrapolate the historical data 410 to populate unknown properties and information in the historical data 410. The extrapolation module 434 may use the historical data 410 to populate unknown properties and information in the historical data 410 by borrowing and extrapolating known data from data sets. The extrapolation module 434 may also use new data input into the historical data 410 to further refine its extrapolations and may periodically update the extrapolated data based on new input data. This process over time may further refine the model and allow better correlations to be developed in the extrapolation module 434.

In some embodiments, the extrapolation module 434 may use the input data 450 and the historical data 410 to extrapolate the information in input data 450 to fill in any information missing from the input data 450. By extrapolating the input data, the model may be better able to identify information in the historical data 410 that may be relevant to the input data 450.

The applicability index module 438 may be used to track and calculate the applicability of any recommended scale inhibitors to the associated water-bearing fluid. For example, the applicability index module 438 may prepare a numerical representation of the performance or applicability of the scale inhibitor based on the performance data 416 from the historical data 410. In some embodiments, the applicability index module 438 may further determine or identify the compatibility of a selected scale inhibitor with the target water-bearing fluid.

The selection module 440 may select the output data 460 selected by the model 430 that meets a predetermined set of criteria, such as compatibility and/or applicability index criteria. The selection module 440 may organize the output data 460. For example, the selection model 440 may organize the output data 460 with the highest scale applicability index first. In some examples, the selection model 440 may organize the output data 460 in any other manner, such as by price, availability, location of a stockpile of the scale inhibitor, amount of scale inhibitor on site, and so forth.

Other modules 444 may also be used by the model 430. For example, a mapping module may use the source location data and geological information of a water-bearing fluid to collate data sets that may share or be near the source location and may be from the same geological formation. The mapping module may identify the geographic limits of different water-bearing fluid formations that can be used by the system 400 or model 430 to group together or collate the historical data 410. Using the associated performance data, the water-bearing fluids properties 414, and scale inhibitors properties 412 may be similar enough to allow the model to make high confidence level recommendations of scale inhibitors that will work effectively with the water-bearing fluid produced from that water-bearing fluid formation because of a larger available data set.

Other modules 444 may also include a communication module (not shown) allowing the model 430 to communicate with historical data 410 that may be stored in the cloud, remote servers, or other remote computing devices. A communication module may also be used to allow remote access by users of the model 430. Additional modules may include a search module that searches for data that may be used to update the historical data 410.

Input data 450 may be provided to the model 430 including select target water-bearing fluid properties 452, inhibitor properties 454, use conditions 455, and user conditions 456. A user may want specific information about a scale inhibitor or type of scale inhibitor from the model 430 and so may input select inhibitor properties 454. The information that a user may provide for the select inhibitor properties 454 may include the name of a specific scale inhibitor, its composition, the chemical structure of specific molecules used in the scale inhibitor, such as chain length, length of branches, etc. Further, the select inhibitor properties 454 may limit output to scale inhibitors above or below a threshold related but not limited to a scale inhibition efficiency, price, dosage, or availability.

The target water-bearing fluid properties 452 may include the name or other identifier of the target water-bearing fluid, a description of the source location, such as a geographic description, address, global positioning system coordinates, sections in a government survey system, the name or description of the geological formation that the target water-bearing fluid may be sourced from, and other geographically related properties of the target water-bearing fluid properties 452.

Use conditions 455 may also be described by a user. Use conditions 455 include but are not limited to environmental and field operating conditions. For example, use conditions 455 may include water-bearing fluid bulk temperature, pipeline outer wall temperature, weather conditions, flow rates, and pipeline and equipment materials and associated surface friction.

The user conditions 456 may specify the output data 460 that the user wants from the model. For example, the user may only want data, extrapolations, and predictions that have an applicability index above a certain threshold or scale inhibitors that are compatible with the fluid properties 452. The user may also specify how the recommendations should be ordered; alphabetically, by confidence level, by scale inhibition efficiency, by similarity to the target water-bearing fluid, etc.

The model 430 may use an artificial intelligence model or a machine learning model to correlate the input data 450, such as the target water-bearing fluid properties 452 with water-bearing fluid properties 414 of the historical data 410. In one embodiment, the model 430 may select a numerical tolerance to identify water-bearing fluids in the historical data 410 that may be similar to the target water-bearing fluid properties. For example, the model 430 may provide a 1 percent tolerance for weight percent for scales in a water-bearing fluid in seeking to identify similar water-bearing fluids in the historical data 410. Further, the model 430 may select a second or more properties to apply a numerical tolerance to for the identification of similar water-bearing fluids. These parameters may facilitate the identification of data that may be used to identify scale inhibitors having a high predicted scale inhibitor efficiency with the target water-bearing fluid.

The model 430 may then use the correlated water-bearing fluid properties 414 to identify performance data 416, scale inhibitor properties 412, field information 418, and other information 419 within the historical data 410. The model 430 may organize and output similar water-bearing fluid properties 462, recommended scale inhibitors 464 and their properties 466, the associated performance data 468, and other information 470 that may have been deemed relevant by the model 430 or requested by a user. As previously discussed above, the model 430 may extrapolate historical data 410 to identify unknown parameters in a data set of the historical data 410 to output data including one or more of a list of water-bearing fluids having one or more properties within a numerical tolerance of the properties of the target water-bearing fluid, a list of scale inhibitors for use with the target water-bearing fluid; and a list of inhibitor properties whose inhibitor effectiveness is above a threshold.

The output data 460 may potentially include a custom scale inhibitor not based on a currently available inhibitor. For example, the model 430 may recommend mixing 4 or more known scale inhibitors at a specific ratio or percentage in order to achieve a superior scale inhibitor efficiency. Alternatively, the output data 460 may include a set of properties for a custom scale inhibitor based on the correlations identified by the model 430.

The output data 460 may provide the inhibitor properties whose scale inhibitor effectiveness is above a numerical threshold, which may be provided by the user or predetermined. The model may also use parameter importance established during model building, where the parameter importance indicates dependence of inhibitor applicability index or compatibility on the parameter, and the model may define a parameter importance score based on the parameter importance. Parameters may refer to the properties of the scale inhibitors, water-bearing fluids, and performance data that the model 430 correlates with a predicted high scale inhibitor efficiency for a particular water-bearing fluid.

Further, the model 430 may perform response surface modeling or other sensitivity analysis to determine variables of inhibitor structure to which inhibition effectiveness is most effective for a target water-bearing fluid. Additionally, or alternately, parameter importance to the applicability index can be identified during model building and used in design or procurement of scale inhibitors.

In accordance with at least one embodiment of the present disclosure, the system 400 may further include a physics-based model 415. The physics-based model 415 may analyze water properties, such as the fluid properties 452 and/or the water-bearing fluids properties 414, and identify certain additional parameters. For example, the physics-based model 415 may include a scale ID module 472 and a saturation index module 474. The physics-based model 415 may analyze the water properties and identify a scale type and a saturation index for the scale type. In some embodiments, the physics-based model 415 may identify multiple scale types and associated saturation indexes for the same set of water properties. This may result in one or more derived features 476 that may be used by the model 430 during training and/or analysis. In some embodiments, the physics-based model 415 may generate the scale type and saturation index for one or more sets of water-bearing fluids properties 414 in the historical data 410.

The model 430 may also base its recommendations on parameters such as a product catalog 422 or other market data available in the field information 418. For example, the model 430 may output a recommendation that notes the cost of precursors, or manufacturing methods and or equipment for a particular scale inhibitor may be unusually expensive or difficult to obtain or use so that implementation would be cost prohibitive. The target water-bearing fluid properties 452 may be input to include a cost threshold that may instruct the model 430 to remove potential scale inhibitors from the output if the cost to produce or the precursors are sold at a price high enough to make the use of that scale inhibitor cost prohibitive in spite of having a high scale inhibiting effectiveness and confidence level. Similarly, the dosage of a scale inhibitor may be used to increase or decrease the overall applicability index or confidence level or placement of the scale inhibitor in the listing of recommendations if the dosage is too high to be economically feasible.

The model 430 may limit recommendations to off-the-shelf scale inhibitors that are currently stocked in sufficient quantities by commercial entities that regularly upload their inventory data to the historical data 410. In other cases, the model 430 may output water-bearing fluids similar to the target water-bearing fluid, based on a numerical score computed from numerically described properties of the input water-bearing fluid, along with all inhibitors tested with that water-bearing fluid in the historical data 410, and their test results. The model 430 may be configured to output a scale inhibitor functionality type predicted to be most effective for use with the target water-bearing fluid.

Once the model 430 has output data 460, a user may test one or more of the scale inhibitor recommendations with the target water-bearing fluid 480 to determine its actual scale inhibition efficiency. Alternatively, the model 430 may also specify testing parameters as part of the output data to validate the model's extrapolations and predictions. The resulting test data may be used to make a final selection of the scale inhibitor and associated dosage 482. Further, the resulting test data may be input into the historical data 410. The new data may be used to recalibrate and re-train the model 430. The historical data 410 may also be used to update the model 430 on a regular periodic basis or only when a certain threshold of new data or certain types of new data are added to the historical data 410. For example, the model 430 may be recalibrated and re-trained when test data for previously recommended scale inhibitors for a target water-bearing fluid 480 are input into the historical data 410 or when the final selection of a scale inhibitor and its associated dosage for a target water-bearing fluid is made 482 and this information is input into the historical data 410. Alternatively, the model 430 may be recalibrated and re-trained when a report of observations of the actual usage of the scale inhibitor at the recommended dosage in a target water-bearing fluid within equipment is input into the historical data.

To calibrate the model 430, a calibration module 442 may tune the relationships between parameters of the historical data 410 and the model 430 until the output data are consistent with test results 480 from recommended scale inhibitor tests with a target water-bearing fluid. In some embodiments, the parameters of the historical data 410 and the model 430 may be calibrated to minimize the differences between the extrapolated and predicted values versus the actual test results 480.

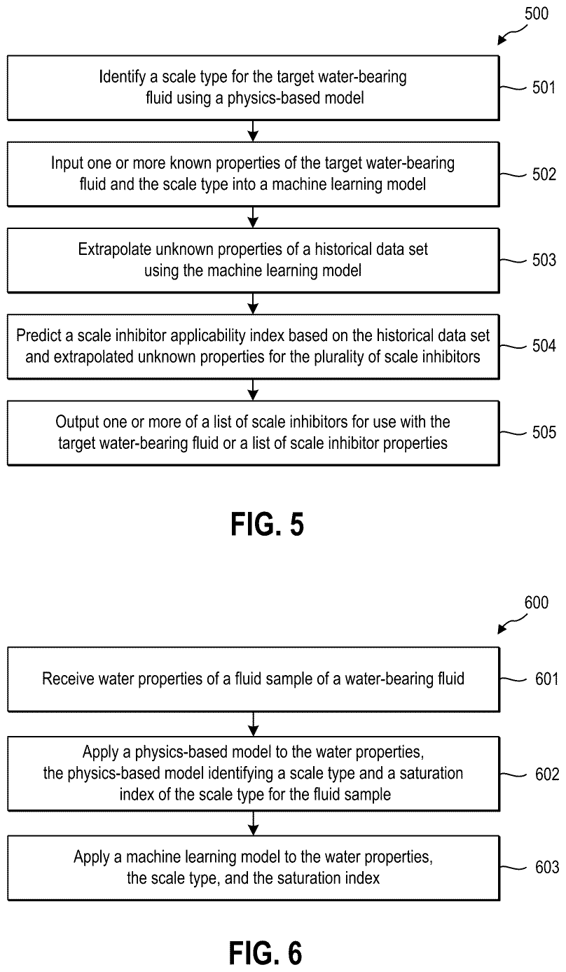

FIG. 5 and FIG. 6, the corresponding text, and the examples provide a number of different methods, systems, devices, and computer-readable media of the scale inhibitor selection system. In addition to the foregoing, one or more embodiments can also be described in terms of flowcharts comprising acts for accomplishing a particular result, as shown in FIG. 5 and FIG. 6. FIG. 5 and FIG. 6 may be performed with more or fewer acts. Further, the acts may be performed in differing orders. Additionally, the acts described herein may be repeated or performed in parallel with one another or parallel with different instances of the same or similar acts.

As mentioned, FIG. 5 illustrates a flowchart of a series of acts or a method 500 for selecting scale inhibitors for a target water-bearing fluid, according to at least one embodiment of the present disclosure. While FIG. 5 illustrates acts according to one embodiment, alternative embodiments may omit, add to, reorder, and/or modify any of the acts shown in FIG. 5. The acts of FIG. 5 can be performed as part of a method. Alternatively, a computer-readable medium can comprise instructions that, when executed by one or more processors, cause a computing device to perform the acts of FIG. 5. In some embodiments, a system can perform the acts of FIG. 5.

A scale inhibitor selection system may identify a scale type for a target water-bearing fluid using a physics-based model at 501. The scale inhibitor selection system may input one or more known properties of the target water-bearing fluid and the scale type into a machine learning model at 502. The scale inhibitor selection system may extrapolate unknown properties of a historical data set using the machine learning model at 503. The machine learning model is trained on the historical data set including one or more properties of a plurality of water-bearing fluids, one or more properties of a plurality of scale inhibitors to inhibit the scale type identified by the physics-based model, and one or more scale inhibitor applicability indexes of the scale inhibitors with the plurality of water-bearing fluids. The machine learning model may predict a scale inhibitor applicability index based on the historical data set and extrapolated unknown properties for the plurality of scale inhibitors at 504. The machine learning model may output one or more of a list of scale inhibitors for use with the target water-bearing fluid or a list of scale inhibitor properties at 505.

As mentioned, FIG. 6 illustrates a flowchart of a series of acts or a method 600 for selecting scale inhibitors for a target water-bearing fluid, according to at least one embodiment of the present disclosure. While FIG. 6 illustrates acts according to one embodiment, alternative embodiments may omit, add to, reorder, and/or modify any of the acts shown in FIG. 6. The acts of FIG. 6 can be performed as part of a method. Alternatively, a computer-readable medium can comprise instructions that, when executed by one or more processors, cause a computing device to perform the acts of FIG. 6. In some embodiments, a system can perform the acts of FIG. 6.

A scale inhibitor selection system may receive water properties of a fluid sample of a water-bearing fluid at 601. A physics-based model may be applied to the water properties at 602. The physics-based model may identify a scale type and a saturation index of the scale type for the fluid sample. A machine learning model may be applied to the water properties, the scale type, and the saturation index at 603. The machine learning model may be trained to generate an output of at least one scale inhibitor based on an input of water properties. The machine learning model outputs a list of scale inhibitors for the fluid sample based on the water properties, the scale type and the saturation index.

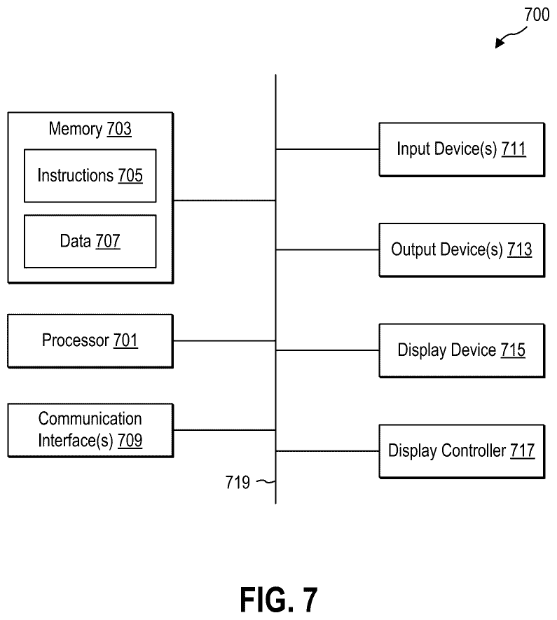

FIG. 7 illustrates certain components that may be included within a computer system 700. One or more computer systems 700 may be used to implement the various devices, components, and systems described herein.

The computer system 700 includes a processor 701. The processor 701 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 701 may be referred to as a central processing unit (CPU). Although just a single processor 701 is shown in the computer system 700 of FIG. 7, in an alternative configuration, a combination of processors (e.g., an ARM and DSP) could be used.

The computer system 700 also includes memory 703 in electronic communication with the processor 701. The memory 703 may be any electronic component capable of storing electronic information. For example, the memory 703 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 705 and data 707 may be stored in the memory 703. The instructions 705 may be executable by the processor 701 to implement some or all of the functionality disclosed herein. Executing the instructions 705 may involve the use of the data 707 that is stored in the memory 703. Any of the various examples of modules and components described herein may be implemented, partially or wholly, as instructions 705 stored in memory 703 and executed by the processor 701. Any of the various examples of data described herein may be among the data 707 that is stored in memory 703 and used during execution of the instructions 705 by the processor 701.

A computer system 700 may also include one or more communication interfaces 709 for communicating with other electronic devices. The communication interface(s) 709 may be based on wired communication technology, wireless communication technology, or both. Some examples of communication interfaces 709 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 700 may also include one or more input devices 711 and one or more output devices 713. Some examples of input devices 711 include a keyboard, mouse, microphone, remote control device, button, joystick, trackball, touchpad, and lightpen. Some examples of output devices 713 include a speaker and a printer. One specific type of output device that is typically included in a computer system 700 is a display device 715. Display devices 715 used with embodiments disclosed herein may utilize any suitable image projection technology, such as liquid crystal display (LCD), light-emitting diode (LED), gas plasma, electroluminescence, or the like. A display controller 717 may also be provided, for converting data 707 stored in the memory 703 into text, graphics, and/or moving images (as appropriate) shown on the display device 715.

The various components of the computer system 700 may be coupled together by one or more buses, which may include a power bus, a control signal bus, a status signal bus, a data bus, etc. For the sake of clarity, the various buses are illustrated in FIG. 7 as a bus system 719.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for selecting scale inhibitors for a target water-bearing fluid, the method comprising:
identifying a scale type for the target water-bearing fluid using a physics-based model;
inputting one or more known properties of the target water-bearing fluid and the scale type into a machine learning model;
extrapolating unknown properties of a historical data set using the machine learning model, the machine learning model trained on the historical data set including one or more properties of a plurality of water-bearing fluids, one or more properties of a plurality of scale inhibitors to inhibit the scale type identified by the physics-based model, and one or more scale inhibitor applicability indexes of the scale inhibitors with the plurality of water-bearing fluids;
predicting a scale inhibitor applicability index based on the historical data set and extrapolated unknown properties for the plurality of scale inhibitors; and
outputting one or more of a list of scale inhibitors for use with the target water-bearing fluid or a list of scale inhibitor properties.

2. The method of claim 1, further comprising selecting or designing an output scale inhibitor, based on the output, for use with the target water-bearing fluid.

3. The method of claim 2, further comprising:
validating the scale inhibitor applicability index by testing the scale inhibitor with the target water-bearing fluid; and
adding results of the testing to the historical data set.

4. The method of claim 1, further comprising calculating a scale inhibitor efficiency value for each combination of a scale inhibitor and the target water-bearing fluid using the machine learning model and the historical data set.

5. The method of claim 4, further comprising ordering the scale inhibitors for use with the target water-bearing fluid based on the scale inhibitor applicability index.

6. The method of claim 1, wherein identifying the scale type includes identifying a risk of scale formation of the scale type.

7. The method of claim 6, wherein identifying the risk of scale formation includes identifying a saturation index of the formation of the scale type.

8. The method of claim 7, wherein predicting the scale inhibitor applicability index is based at least partially on the saturation index.

9. The method of claim 1, further comprising generating a dose for one or more of the list of scale inhibitor properties.

10. The method of claim 1, wherein identifying the scale type includes inputting the one or more known properties of the target water-bearing fluid into the physics-based model.

11. The method of claim 1, wherein predicting the scale inhibitor applicability index includes correlating the scale inhibitor applicability index with a range of values representing the water-bearing fluid properties and scale inhibitor structural data.

12. The method of claim 1, wherein the one or more properties of a plurality of water-bearing fluids includes a location of a source of one of the plurality of water-bearing fluids.

13. The method of claim 1, wherein the machine learning model outputs a compatibility rating of the list of scale inhibitors and the target water-bearing fluid.

14. A method comprising:
receiving water properties of a fluid sample of a water-bearing fluid;
applying a physics-based model to the water properties, the physics-based model identifying a scale type and a saturation index of the scale type for the fluid sample; and
applying a machine learning model to the water properties, the scale type, and the saturation index, the machine learning model trained to generate an output of at least one scale inhibitor based on an input of water properties, wherein the machine learning model outputs a list of scale inhibitors for the fluid sample based on the water properties, the scale type and the saturation index.

15. The method of claim 14, further comprising selecting or designing a chosen scale inhibitor, based on the output, for use with the fluid sample.

16. The method of claim 15, further comprising testing the chosen scale inhibitor with the fluid sample.

17. The method of claim 16, further comprising training the machine learning model based on testing the chosen scale inhibitor.

18. The method of claim 14, wherein receiving the water properties of the fluid sample includes receiving field operational parameters, and wherein applying the physics-based model includes applying the physics-based model to the field operational parameters to identify the scale type and the saturation index based on the field operational parameters.

19. The method of claim 14, further comprising generating an applicability index for each of the list of scale inhibitors, the list of scale inhibitors ranked by the applicability index.

20. A system, comprising:
a processor; and
memory, the memory including instructions that cause the processor to:
identify a scale type for target water-bearing fluid using a physics-based model;
input one or more known properties of the target water-bearing fluid and the scale type into a machine learning model;
extrapolate unknown properties of a historical data set using the machine learning model, the machine learning model trained on the historical data set including one or more properties of a plurality of water-bearing fluids, one or more properties of a plurality of scale inhibitors to inhibit the scale type identified by the physics-based model, and one or more scale inhibitor applicability indexes of the scale inhibitors with the plurality of water-bearing fluids;
predict a scale inhibitor applicability index based on the historical data set and extrapolated unknown properties for the plurality of scale inhibitors; and
output one or more of a list of scale inhibitors for use with the target water-bearing fluid or a list of scale inhibitor properties.
